(19) United States
(12) Plant Patent Application Publication
(10) Pub. No.: US 2010/0083412 P1
(43) Pub. Date: Apr. 1, 2010

(54) Cosmos plant named 'CC3013/01'

(50) ;

(76) Inventors: Russell Poulter, Dunedin (NZ); Keith Hammett, Auckland (NZ)

Correspondence Address: PLANT HAVEN, INC., P.O. BOX 3056, SANTA BARBARA, CA, 93130, US

(21) Appl. No.: 12/587,072

(22) Filed: Sep. 30, 2009

(30) Foreign Application Priority Data
Oct. 1, 2008 (NZ) HOM254

(51) Int. Cl. A01H 5/00

(52) U.S. Cl. PLT/418

(57) ABSTRACT

A new cultivar of Cosmos plant named ‘CC3013/01’ which exhibits a compact habit and which bears dark maroon colored flowers from late spring through late summer. In combination these traits set ‘CC3013/01’ apart from all other existing varieties of Cosmos known to the inventor.

### GENUS

COSMOS

### SPECIES

×atrosanguineus

### DENOMINATION

‘CC3013/01’

### BACKGROUND OF THE INVENTION

This application claims the benefit of priority under 35 U.S.C. 119(f) of the application for a grant of New Zealand Plant Breeders Rights which was filed for the instant plant variety on Oct. 1, 2008, Application Number HOM254.

The present invention relates to a new and distinct cultivar of Cosmos, also commonly known as chocolate cosmos, that is grown for use as a flowering plant for pots and containers and for planting in the garden and landscape. The new cultivar is known botanically as Cosmos×atrosanguineus and will be referred to hereinafter by the cultivar name ‘CC3013/01’.

‘CC3013/01’ is a complex hybrid plant that is the product of a breeding program which is conducted jointly by the inventors. Pollinations are carried out in a cultivated area in Dunedin, New Zealand and evaluations and selections are carried out in Auckland, New Zealand.

‘CC3013/01’ resulted from the open pollination between Cosmos ‘Pinot Noir’ (unpatented) as female parent plant and an unknown, unreleased seedling as male parent plant.

The inventor selected ‘CC3013/01’ in 2005 for its exceptional compactness which distinguishes ‘CC3013/01’ from both parents and from all other varieties or seed-raised plants known to the inventors.

Asexual reproduction of ‘CC3013/01’ was first accomplished under the inventors' direction at a tissue culture laboratory in Auckland, New Zealand. The method of asexual propagation used was division by tissue culture. The inventors have grown out successive generations of plants resulting from the tissue culture and have determined that the characteristics of ‘CC3013/01’ are stable and reproduced true to type in successive generations of asexual reproduction.

### SUMMARY OF THE INVENTION

The following traits have been repeatedly observed and represent the characteristics of the new Cosmos cultivar ‘CC3013/01’. These traits in combination distinguish this cultivar from all other varieties of Cosmos known to the inventor. ‘CC3013/01’ has not been tested under all possible conditions and phenotypic differences may be observed with variations in environmental, climatic and cultural conditions, however, without any variance in genotype.
1. Cosmos ‘CC3013/01’ exhibits a compact habit.
2. Cosmos ‘CC3013/01’ exhibits flowers that are dark maroon in color.
3. Cosmos ‘CC3013/01’ blooms from late spring through late summer.
4. After one year of growth, Cosmos ‘CC3013/01’ achieves a height (including flowers) of 0.5 m and a spread of 0.5 m.
5. Cosmos ‘CC3013/01’ is a tender perennial which is hardy in USDA Hardiness Zone 8 and above.

### BRIEF DESCRIPTION OF THE DRAWINGS

The accompanying color drawings illustrate the overall appearance of the new Cosmos variety ‘CC3013/01’ showing colors as true as it is reasonably possible to obtain in colored reproductions of this type. Colors in the drawings may differ from the color values cited in the detailed botanical description, which accurately describe the actual colors of the new variety ‘CC3013/01’.

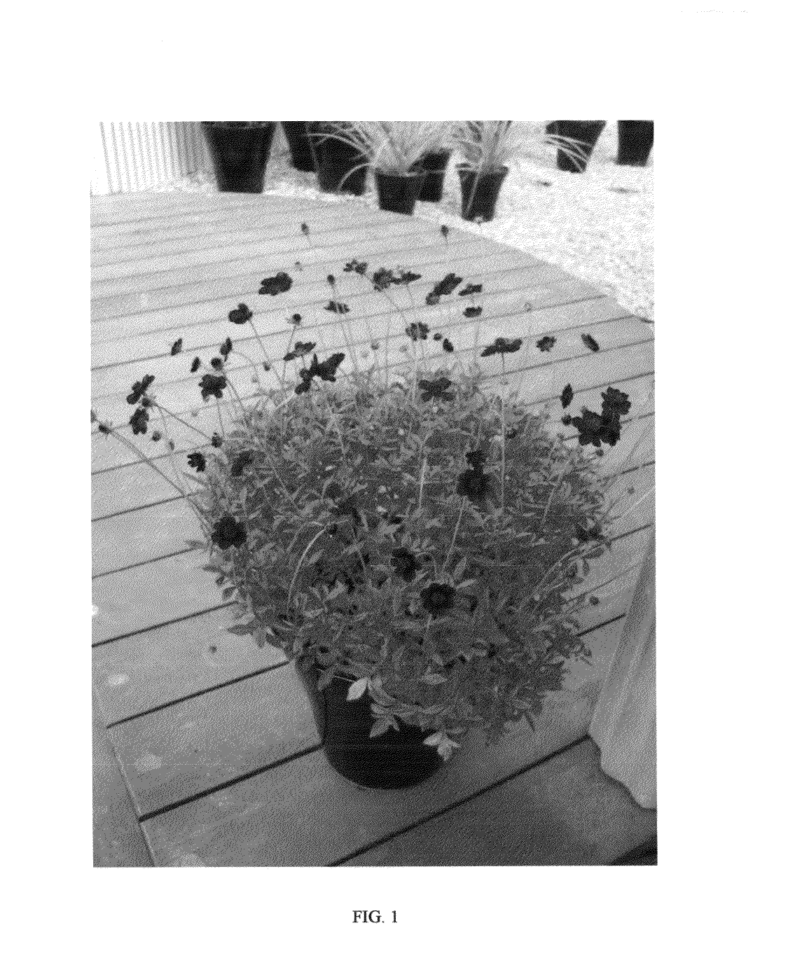

The drawing labeled as FIG. 1 depicts an entire flowering plant.

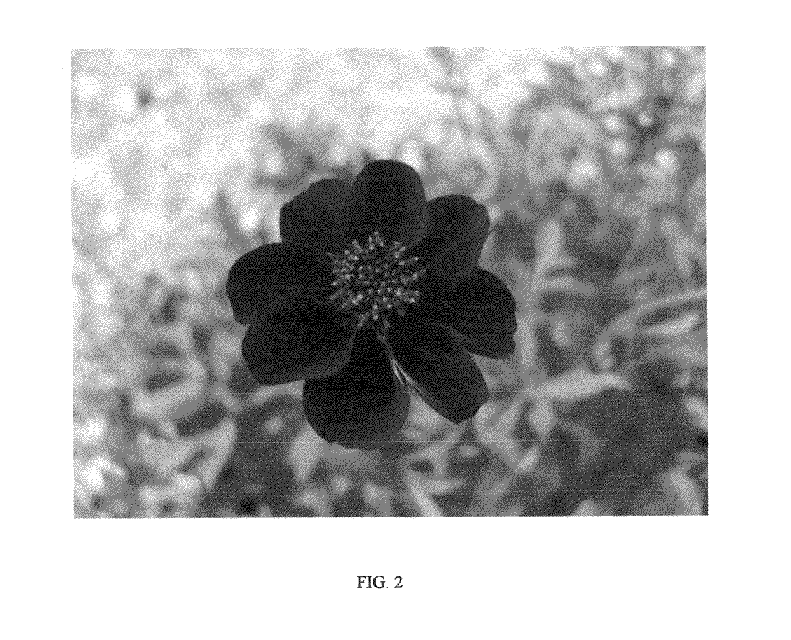

The drawing labeled as FIG. 2 depicts a close-up view of the flower.

Both drawings were made from one year old plants which have been grown in 1.5-litre containers in Auckland, New Zealand. No chemicals were used to treat the plants.

### DETAILED BOTANICAL DESCRIPTION

The following is a detailed description of the new cultivar ‘CC3013/01’. Data was collected in summer in New Zealand from 1.5-litre container plants which had been grown out of doors in Auckland, New Zealand. The color determinations are in accordance with the 2001 edition of The Royal Horticultural Society Colour Chart, London, England, except where general color terms of ordinary dictionary significance are used. No chemicals were used to treat the plants. Growing conditions are typical to other Cosmos.
Botanical Classification:
Family: Asteraceae.
Genus: Cosmos.
Species: ×atrosanguineus.
Denomination: ‘CC3013/01’.
Parentage:
   Male parent.—Unknown.
   Female parent.—Cosmos ‘Pinot Noir’ (unpatented).
Plant Type: Flowering perennial.
Use of plant: As a flowering plant in containers, planters, pots and the garden and landscape.
Bloom period: Late spring through late summer.
Plant habit: Multiple stems arising from the tuber at ground level.
Height of plant: 0.5 m at maturity.
Width of plant: 0.5 m at maturity.
Hardiness: USDA Hardiness Zone 8.
Root system: Initially fibrous, subsequently producing root tubers.
Branching: Basal branching and further branching from the axils of the lower leaves.
Propagation method: Tissue culture.
Cultural requirements: Grow in full sun, in well drained and moderately fertile soil, keep well fertilized and watered.
Diseases and pests: No particular resistance or susceptibility to diseases and pests are known to the inventors.
Crop time: 3 months starting in the spring and 6 months starting in the fall.
Plant stem:
   Shape.—Cylindrical.
   Length (basal stems).—0.3 m.
   Diameter (basal stems).—8 mm at base, 5 mm towards apex.
   Length (axillary stems).—0.2 m.
   Diameter (axillary stems).—4 mm-5 mm.
   Surface texture (all stems).—Sparely hirsute.
   Color.—Crimson, 187A.
   Internode length (between nodes).—30 mm-70 mm.
Leaf:
   Type.—Evergreen.
   Shape.—Pinnate.
   Apex.—Acute.
   Base.—Acuminate.
   Venation.—Pinnate.
   Vein color.—138A.
   Margin.—Entire.
   Arrangement.—Opposite.
   Surface texture (upper surface).—Smooth.
   Surface texture (lower surface).—Minutely Rugose.
   Dimensions.—Length: 110 mm to 150 mm Width: 40 to 60 mm.
   Color.—Upper surface: 138A Lower surface: 138C.
   Attachment.—Petiolate.
   Petiole dimensions.—Length: 50 mm-75 mm Diameter: 2 mm-4 mm.
   Petiole texture.—Smooth.
   Petiole color.—138A.
Inflorescence:
   Type.—Capitulum.
   Fragrance.—Pleasant: reminiscent of chocolate.
   Flowering season.—Late spring through late summer.
   Persistence of inflorescence on and off plant.—7-10 days.
   Quantity of inflorescences per plant at peak summer flowering.—many: approximately 25 flowers in bud to fully open.
Peduncles:
   Length.—40 mm-45 mm, diameter 2 mm.
   Angle to stem.—Vertical initially, arching away.
   Strength.—Wiry.
   Texture.—Smooth.
   Color.—Predominantly 138A with longitudinal striations of red 187B.
Buds:
   Shape.—Globose.
   Dimensions.—Length 9.5 mm, width 9.5 mm.
   Color.—138A.
Ray florets:
   Arrangement.—Single outer circle.
   Length.—15 mm-17 mm.
   Width.—12 mm-14 mm.
   Apex.—Truncate.
   Base.—Obtuse.
   Margin.—Entire.
   Texture (upper surface).—Puberulous.
   Texture (lower surface).—Puberulous, slight pubescence on veins.
   Number of ray florets per inflorescence.—6-8.
   Color (both surfaces).—Dark maroon, darker than 187A.
Disc florets:
   Arrangement.—Circular.
   Shape.—Tubular.
   Length at anthesis.—17 mm.
   Width at anthesis.—1.5 mm.
   Apex.—Divergent stigmas.—top of tube 5-lobed, acute.
   Base.—Subtended directly from receptacle.
   Margin.—Smooth.
   Texture, upper and lower surfaces of tube.—Smooth.
   Number of ray florets per inflorescence.—6-8.
   Color (both surfaces).—Dark maroon, darker than 187A.
Reproductive organs:
   Androecium.—Present on disc florets only.
   Anthers.—Shape: Flattened cylinder, dehiscing on the inner surface only Length: 4 mm-5 mm Color: Dark maroon, darker than 187A.
   Pollen.—Copious, colored yellow 17A.
   Gynoecium.—Present on disc florets only.
   Pistil length.—13 mm.
   Stigma shape.—Divergent bifid curved.
   Stigma color.—Dark maroon, darker than 187A.
   Style length.—10 mm.
   Style color.—Cream 157D at base, dark maroon at tip, darker than 187A.
   Ovary shape.—Cylindrical, length 3 mm, width 0.75 mm.
   Ovary color.—Cream 155D.
   Seed.—Not Present.

1. A new and distinct cultivar of Cosmos plant named ‘CC3013/01’ as described and illustrated herein.
